(19) United States
(12) Patent Application Publication
(10) Pub. No.: US 2006/0200079 A1
(43) Pub. Date: Sep. 7, 2006

(54) Drainage catheter

(76) Inventor: Anders Magnusson, Uppsala (SE)

Correspondence Address: FAY KAPLUN & MARCIN, LLP, 15O BROADWAY, SUITE 702, NEW YORK, NY, 10038, US

(21) Appl. No.: 11/069,634

(22) Filed: Mar. 1, 2005

(51) Int. Cl. A61M 5/178

(52) U.S. Cl. 604/164.1; 604/170.03; 604/544; 604/164.13

(57) ABSTRACT

The present invention relates to a catheter comprising a tubular body, a first connector piece, at least one slidable member and at least one elongate member. The first connector piece is permanently attached to the tubular body. The first connector piece and each of the at least one slidable member are arranged to be positioned outside the body. One of the at least one slidable member comprises a second connector piece connectable to the first connector piece. Each of the at least one elongate member has at least one proximal attachment part and a distal coupling part. The distal coupling part of each elongate member is coupled to the distal region of the tubular body and each of the at least one proximal attachment part is attached to one of the at least one slidable member at a proximal attachment point. Each elongate member extends from each respective proximal attachment point to the distal coupling part.

### TECHNICAL FIELD

The present invention relates to medical devices for positioning in internal body cavities such as the collecting system in the kidney, the urinary bladder, etc. In particular, the invention relates to catheters for drainage purposes and means for securing such catheters in place.

### BACKGROUND

In many medical conditions, it is necessary to drain or empty internal body cavities of liquids, e.g., urine, blood, etc. For this purpose, numerous designs of catheters are available and commonly used. If it is required that the drainage is performed over an extended period of time, such as several days, weeks or even months, it is essential that the catheter is properly secured inside the body cavity that is to be drained. One common type of securable catheter is known as a lockable “pig-tail” catheter (or “Cope loop” type catheter). This type of catheter is characterized by including means to form a loop (i.e. curl or pig-tail) at its distal end once the catheter has been positioned inside the body cavity, thereby forming an efficient means to prevent or resist the catheter from being pulled out.

Percutaneous nephrostomy is one of the oldest techniques used in interventional radiology. It comprises placing a drainage catheter or tube inside the renal pelvis through a skin insertion. Early methods were very time consuming, requiring multiple procedural steps. In fact, the procedure could take up to a week to complete. However, the technique has been developed substantially and today the entire procedure is performed at one time in one continuous sequence of steps. The drainage catheter used for percutaneous nephrostomy today is typically the above mentioned “pig-tail” type drainage catheter.

One “pig-tail” type drainage catheter and a catheterization kit comprising the catheter and means for securing the catheter in place are described in WO 2004/002563. The catheter in WO 2004/002563 comprises a tubular body, a ring member and an elongate member. The ring member encircles at least a portion of the tubular body and is slidable along the tubular body. The elongate member is coupled to the distal region of the tubular body and attached to the ring member. By pulling the ring member in the direction of a proximal end of the tubular body, the elongate member is stretched at a certain position. Further pulling the ring member in the direction of the proximal end of the tubular body implies that a loop is formed in the distal region of the tubular body since the elongate member is non-elastic.

The catheter in WO 2004/002563 may be used together with an introducer of the catheterization kit in WO 2004/002563. The introducer has a proximal end arranged to be positioned outside the body and a distal end arranged to be positioned inside the body. The catheter is insertable into the introducer, which is introduced into the body cavity before the catheter and is utilized for guidance and protection of the catheter during insertion thereof into a body cavity as well as during a drainage procedure. Furthermore, the introducer is utilized for locking the loop when it has been formed in order to prevent it from reducing its size or being eliminated. The length of the introducer and the length of the elongate member between the attachment to the ring member and the coupling to the distal region are essentially equal. When the catheter is positioned in the introducer such that the ring member abuts the proximal end of the introducer, the elongate member is stretched and the coupling of the elongate member to the distal region of the catheter is positioned just outside the distal end of the introducer. Further advancing the catheter thereafter into the introducer creates a loop at the distal end of the introducer due to that the elongate member is non-elastic. After the loop has been formed, the catheter may be locked by a locking mechanism to the introducer. The locking mechanism prevents the loop from reducing its size or being eliminated during a drainage procedure. The introducer is left in the body during a drainage procedure. WO 2004/002563 is herein incorporated by reference in its entirety.

However, in certain medical conditions, it is desirable to leave as few devices as possible in the body during the drainage procedure. Thus, in such medical conditions, it is not desirable to leave the introducer of the catheterization kit in WO 2004/002563 in the body despite the many advantages with that catheterization kit.

### SUMMARY

It is an object of the present invention to provide a catheter, which has been sought for, combining the obvious advantages of the catheterization kit described above with leaving only a minimum of artificial parts in the patient's body.

This object is achieved by a catheter comprising: a tubular body having a distal region and a proximal region; a first connector piece being permanently attached to the tubular body and being arranged to be positioned outside the body; at least one member being arranged to encircle at least a portion of the circumference of the tubular body and to be slidable along the tubular body, whereby each of the at least one slidable member is arranged to be positioned outside the body and one of the at least one slidable member comprises a second connector piece connectable to the first connector piece; and at least one elongate member, each of which having at least one proximal attachment part and a distal coupling part, the distal coupling part of each elongate member being coupled to the distal region of the tubular body and each of the at least one proximal attachment part being attached to one of the at least one slidable member at a proximal attachment point, whereby each elongate member extends from each respective proximal attachment point to the distal coupling part, whereby the slidable member to which each of the at least one proximal attachment part is attached is slidable to a position adjacent the first connector piece.

Another object of the present invention is to provide a method of securing a catheter in a body cavity by which a minimum of artificial parts are left in the patient's body.

This object is achieved by the method comprising: Inserting a catheter into a body cavity, the catheter comprising a tubular body having a distal region and a proximal region; a first connector piece being permanently attached to the tubular body and being arranged to be positioned outside the body; at least one member being arranged to encircle at least a portion of the circumference of the tubular body and to be slidable along the tubular body, whereby each of the at least one slidable member is arranged to be positioned outside the body and one of the at least one slidable member comprises a second connector piece connectable to the first connector piece, and at least one elongate member, each of which having at least one proximal attachment part and a distal coupling part, the distal coupling part of each elongate member being coupled to the distal region of the tubular body and each of the at least one proximal attachment part being attached to one of the at least one slidable member at a proximal attachment point, whereby each elongate member extends from each respective proximal attachment point to the distal coupling part, whereby the slidable member to which each of the at least one proximal attachment part is attached is slidable to a position adjacent the first connector piece; advancing the catheter into the body cavity until a distal end of the tubular body is positioned at a correct position within the body cavity; stretching the at least one elongate member by adjusting the position of the second connector piece on the tubular body; and bringing the first connector piece and the second connector piece together, whereby a loop is formed in the distal region of the tubular body.

The catheter according to the invention can provide numerous advantages. For example, the catheter can be used to easily and simply form a loop in a body cavity and to easily lock and secure the catheter in place when a loop is formed with a minimum of devices left in the body cavity during the drainage procedure. This considerably reduces the risk of inflammation and/or infection at the site of drainage. In particular, it was surprisingly found that a catheter lacking a sheath permanently positioned around the tubular body of the catheter in the insertion wound was favourable for several specific treatments.

Still other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures described herein.

### DESCRIPTION OF THE DRAWINGS

In the drawings, wherein like reference characters denote similar elements throughout the several views:

FIG. 1a is a side view of a slidable member of a first embodiment of a catheter according to the invention and the catheter according to the first embodiment without the slidable member;

FIG. 1b is a side view of the first embodiment of the catheter;

FIG. 1c is a side view of the first embodiment of the catheter with a loop formed at a distal region;

FIG. 2 is a side view of a set of medical devices comprised in a catheterization kit according to the invention;

FIGS. 3a-f illustrate schematically the procedure for using the set of medical devices of FIG. 2 and the first embodiment of the catheter according to the invention in the renal pelvis;

FIG. 4a is a side view of a second embodiment of the catheter;

FIG. 4b is a side view of the second embodiment of the catheter with a loop formed at a distal region;

FIG. 5a is a side view of a third embodiment of the catheter;

FIG. 5b is a side view of the third embodiment of the catheter with a loop formed at a distal region;

FIG. 6 is a side view of a fourth embodiment of the catheter;

FIG. 7a is a side view of a variant of the third embodiment of the catheter;

FIG. 7b is a side view of a fifth embodiment of the catheter;

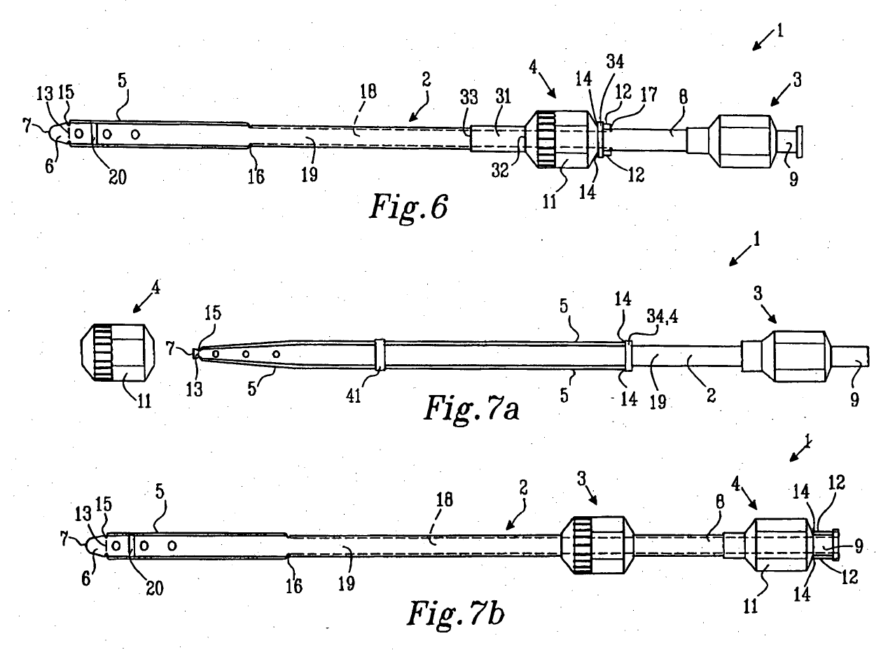

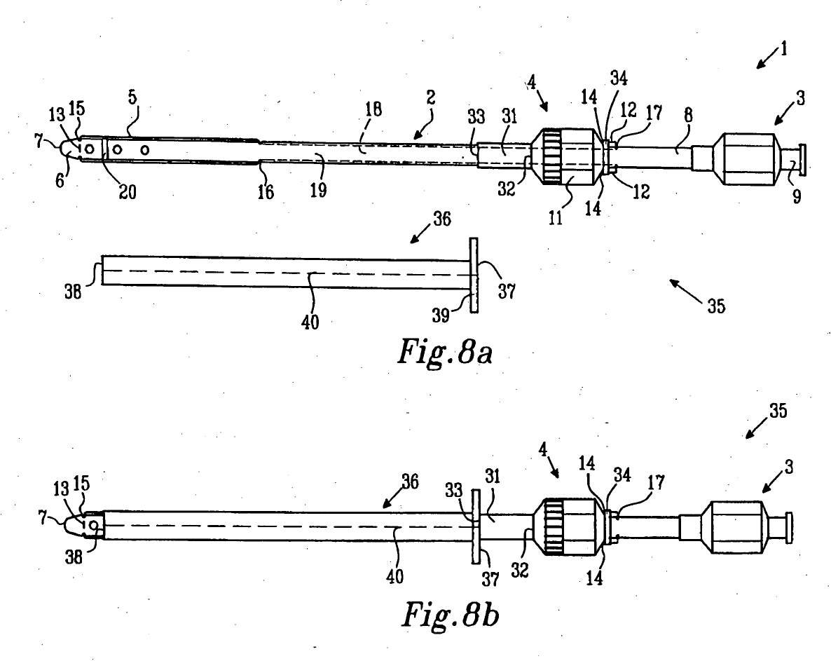

FIGS. 5a-b are side views of a catheterization kit according to the invention, which kit comprises the fourth embodiment of the catheter and a split-away sheath;

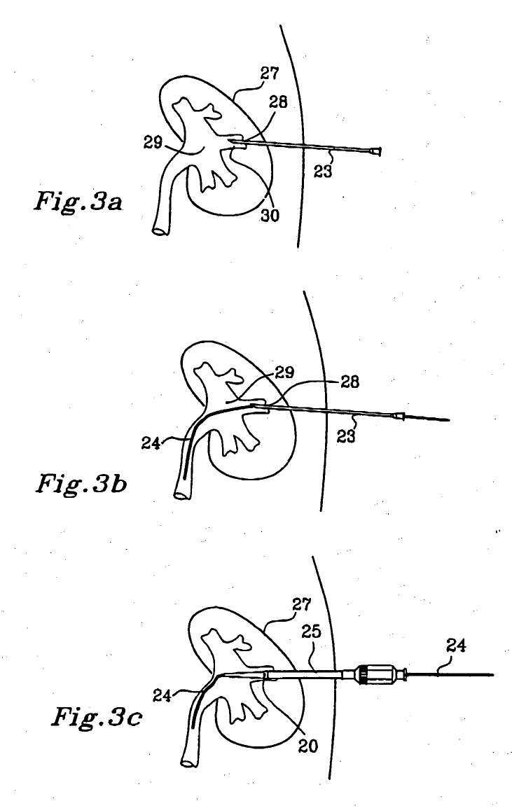

FIGS. 9a-9 illustrate schematically the procedure for using the set of medical devices of FIG. 2 and the catheterization kit of FIGS. 8a-b;

### DETAILED DESCRIPTION

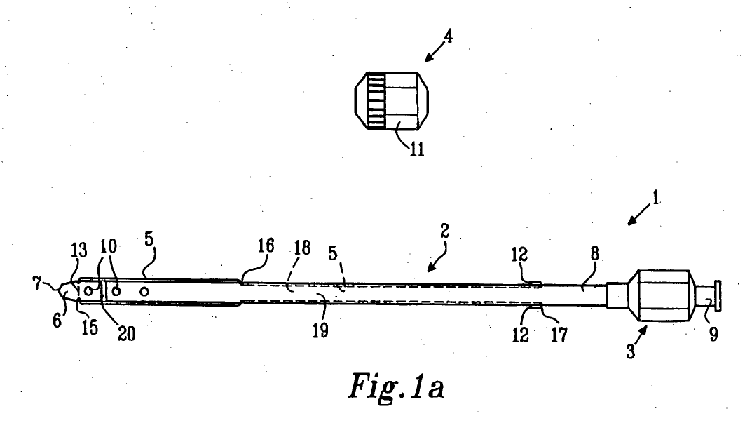

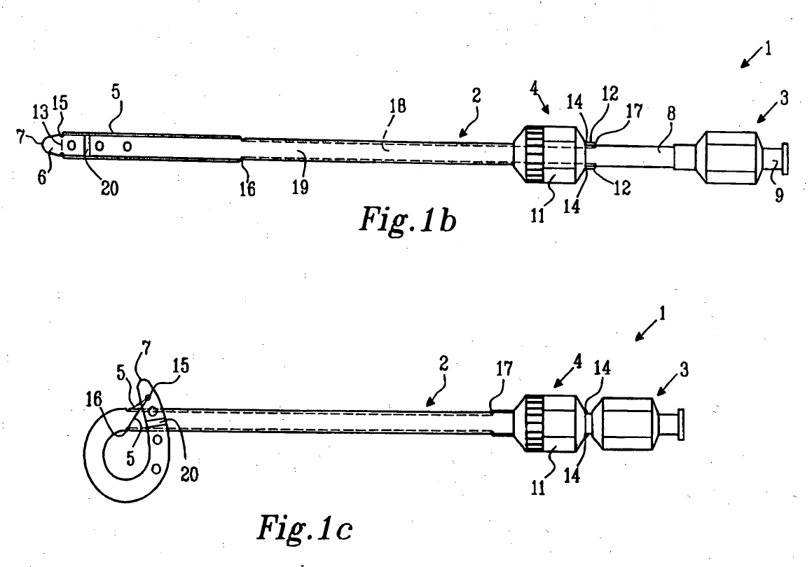

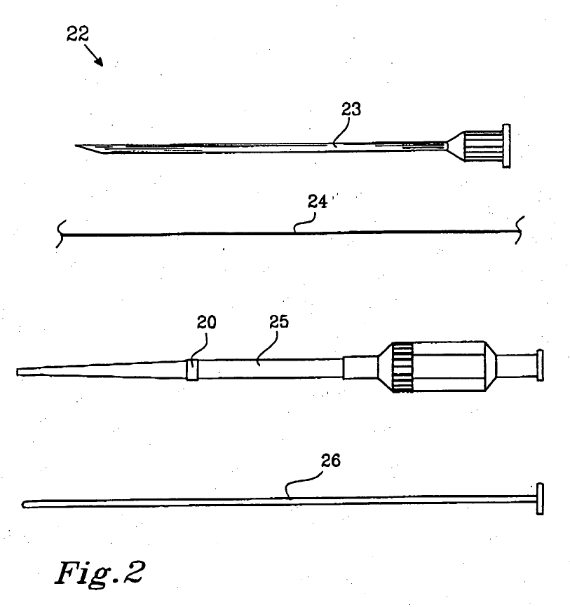

FIGS. 1a-c are side views of a first embodiment of a catheter 1 according to the present invention, which catheter 1 may be used for drainage purposes. The catheter 1 according to the invention is of the “pig-tail” or “Cope loop” type. Thus, it is adapted to form a loop (i.e. curl or pig-tail) within a body cavity in order to resist or prevent the catheter 1 from being pulled out. The catheter 1 comprises a tubular body 2, a first connector piece 3, a slidable member 4 and an elongate member 5.

The tubular body 2 (e.g. a tube) has a distal region 6 with a distal end 7 and a proximal region 8 with a proximal end 9. In the distal region 6, the tubular body 2 includes a plurality of drainage openings 10 for entry into the catheter 2 of drainage liquid, e.g., urine, blood, etc., to be removed.

The terms “proximal” and “distal” are herein used to indicate a location of a part in relation to another part along and/or on an imaginary axis leading from a user, such as a clinician, to the body cavity to be drained during an insertion procedure. The axis may be the imaginary outstretched tubular body. The term “proximal” is used to indicate that a part is located nearer the user during an insertion procedure than a “distal” part, whereas the term “distal” is used to indicate that a part is located further away from the user than a “proximal” part during an insertion procedure. Thus, if a part has a proximal end and a distal end, the proximal end is arranged to be positioned nearer a user than the distal end during an insertion procedure. When any of the terms “proximal” or “distal” is used to denote a part of the catheter 1, they indicate the location of the part in relation to another part during an insertion procedure before any loop has been formed. When a first part is “proximal to” a second part, the first part is in the present context arranged to be positioned nearer a user during an insertion procedure than the second part. Accordingly, when a first part is “distal to” a second part, the first part is in the present context arranged to be positioned more far away from a user during an insertion procedure than the second part.

Furthermore, the distal region 6 of the tubular body 2 includes not only the region of the tubular body 2 at the distal end 7, but the region of the tubular body 2 that may be utilized for forming a loop for securing the catheter 1 in a body cavity. The loop formation will be further described below.

The first connector piece 3 (e.g. a hub) is permanently attached to the tubular body 2 and is arranged to be positioned outside the body. Furthermore, when the catheter 1 has been inserted into a body cavity and a loop has been formed in the tubular body 2, the first connector piece 3 is arranged to interact with a further connector piece in order to prevent the loop from reducing its size or being eliminated. Specifically, the first connector piece 3 is arranged to be removably attachable to the further connector piece. This will be further described below.

According to the invention, the catheter 1 comprises at least one member 4 arranged to encircle at least a portion of the circumference of the tubular body 2 and to be slidable along the tubular body 2. Each of the at least one slidable member 4 is arranged to be positioned outside the body. One of the at least one slidable member 4 comprises a second connector piece 11 connectable to the first connector piece 3.

In the first embodiment, the catheter 1 comprises one slidable member 4 and the slidable member 4 consists of the second connector piece 11 (e.g. a hub). The second connector piece 11 may or may not be separable from the tubular body 2. Furthermore, in the first embodiment, the second connector piece 11 is arranged to be positioned distal to the first connector piece 3 along the tubular body 2. However in variants of the first embodiment described below, the second connector piece 11 is arranged to be positioned proximal to the first connector piece 3. Furthermore, in the first embodiment, the second connector piece 11 encircles the complete circumference of the tubular body 2. However, in variants of the first embodiment, it may encircle only a portion of the circumference of the tubular body 2.

Furthermore, according to the invention, the catheter 1 comprises at least one elongate member 5. Each of the at least one elongate member 5 has at least one proximal attachment part 12 and a distal coupling part 13. The distal coupling part 13 of each elongate member 5 is coupled to the distal region 6 of the tubular body 2. Each of the at least one proximal attachment part 12 is attached to one of the at least one slidable member 4 at a proximal attachment point 14. Each elongate member 5 extends from each respective proximal attachment point 14 to the distal coupling part 13. The slidable member 4, to which each of the at least one proximal attachment part 12 is attached, is slidable to a position adjacent the first connector piece 3.

In the first embodiment, the catheter 1 comprises one elongate member 5 (e.g. a thread) having two proximal attachment parts 12 and one distal coupling part 13, whereby each proximal attachment part 12 constitutes an end of the elongate member 5 and the distal coupling part 13 constitutes a middle region of the elongate member 5. In variants of the first embodiment, however, the proximal attachment parts 12 do not constitute ends of the elongate member 5. Each of the proximal attachment parts 12 is attached to the one slidable member 4, i.e. the second connector piece 11, at a proximal attachment point 14. The proximal attachment points 14 may be positioned anywhere on the second connector piece 11. The attachment of the proximal attachment parts 12 may be provided by means of, for example, a knot, an adhesive or other suitable attaching means. The distal coupling part 13 of the elongate member 5 is coupled to the distal region 6 of the tubular body 2. The coupling of the distal coupling part 13 to the tubular body 2 may be provided at a point near the distal end 7 or at a set distance from the distal end 7. Between the proximal attachment points 14 at the second connector piece 11 and the coupling of the distal coupling part 13 to the distal region 6, the elongate member 5 passes both on the inside and the outside of the tubular body 2, which now will be described in more detail.

In the first embodiment, the tubular body 2 comprises further two first openings 15 in the distal region 6 of the tubular body 2, two second openings 16 that are positioned proximal to the first openings 15 and two third openings 17 that are positioned proximal to the second openings 16. Starting from one of the proximal attachment points 14 at the second connector piece 11, the elongate member 5 passes from the proximal attachment point 14 outside the tubular body 2 to one of the third openings 17, through which it passes into an inner lumen 18 of the tubular body 2. Then the elongate member 5 passes inside the inner lumen 18 to one of the second openings 16, through which it passes out to the outside of the tubular body 2 again. Thereafter the elongate member 5 passes along an outer surface 19 of the tubular body 2 to one of the first openings 15, through which it passes into the inner lumen 18. Next the elongate member 5 passes out of the inner lumen 18 through the other first opening 15 and to the other second opening 16 along the outer surface 19. Thereafter, the elongate member 5 passes to the other third opening 17 along the inner lumen 18 and then outside the tubular body 2 to the other proximal attachment point 14.

Thus, the distal coupling part 13 of the elongate member 5 is in the first embodiment coupled to the distal region 6 of the tubular body 2 by passing through the two first openings 15. Furthermore, the elongate member 5 is generally non-elastic.

The distance between the first openings 15 and the second openings 16 as well as the distance between the second openings 16 and the third openings 17 may be varied. Furthermore, the two first openings 15 may be positioned opposite to each other on the tubular body 2 or not. In addition, one of the two first openings 15 may be positioned proximal to the other first opening 15 along the tubular body 2. The same applies to the two second openings 16 and the two third openings 17.

For purposes of illustration, the one slidable member 4, i.e. the second connector piece 11, according to the first embodiment is shown separated from the tubular body 2 according to the first embodiment in FIG. 1a, whereas the completely assembled catheter 1 according to the first embodiment is shown in FIG. 1b.

The slidable second connector piece 11, to which the elongate member 5 is attached, may be pushed along the tubular body 2 to a position where the elongate member 5 is stretched or taut. If the second connector piece 11 is held steady at that position and the tubular body 2 is advanced such that the first connector piece 3, which is permanently attached to the tubular body 2, is moved in the direction of the second connector piece 11, a loop starts to form in the distal region 6 of the tubular body 2. More specifically, the loop is formed at the coupling of the distal coupling part 13 to the distal region 6 of the tubular body 2, i.e. at the two first openings 15. The loop formation is due to that the elongate member 5 is stretched and cannot be elongated due to its lack of elasticity, whereby the two first openings is can not be further advanced into the body cavity when the first connector piece 3 is moved in the direction of the second connector piece 11. However, when the first connector piece 3 is advanced in the direction of the second connector piece 11, the second openings 16 are advanced and the length of the elongate member 5 extending between the first openings 15 and the second openings 16 is thereby reduced. The first connector piece 3 may be advanced in the direction of the second connector piece 11 until they are adjacent to each other. Thus, the maximum size, or circumference, of the formed loop depends on the distance that the first connector piece 3 may be moved in the direction of the second connector piece 11 when the elongate member 5 is stretched. However, that distance and the distance between the first openings 15 and the second openings 16 must be adjusted to each other such that the desired loop may be formed. Furthermore, the second openings 16 must have such a position on the tubular body 2 such that they may be inserted into a body cavity before the loop formation starts.

The loop may also be formed by moving the slidable second connector piece 11 in the direction of the first connector piece 3 instead of advancing the tubular body 2 by moving the first connector piece 3 in the direction of the second connector piece 11. Thereby, the distal coupling part 13 and, thus, the first openings 15 are pulled in the direction of the second openings 16.

A complete loop is formed when the first connector piece 3 and the second connector piece 11 are adjacent to each other. Then the first connector piece 3 and the second connector piece 11 may be coupled together in a detachable way, e.g. by a thread engagement, a bayonet type lock or by any other suitable locking means. Thereby the catheter 1 may be locked such that the loop is kept at the distal region 6 of the tubular body 2 (FIG. 1c). Furthermore, an O-ring may be placed between the first connector piece 3 and the second connector piece 11 to make the connection leak proof. The use of the catheter 1 for drainage purposes in a body cavity will be further described below.

Furthermore, the tubular body 2 may include a radiopaque section 20. For example, this section can be made of a metal having the desired radiopacity, e.g. platinum (Pt) or gold (Au), although any method and/or material of providing radiopacity is acceptable to provide this function. The position of the radiopaque section 20 on the tubular body 2 is selected such that it enables a physician to ensure under fluoroscopy that the tubular body 2 has been inserted into the collecting system to a certain degree. This will be further described below.

The catheter 1 according to the invention may be comprised in a catheterization kit 21, which further comprises one, several or all of the medical devices in the set of medical devices 22 shown in FIG. 2. The set of medical devices 22 includes a hollow needle 23 (e.g. a cannula or a needle having an inner lumen), a guide wire 24, a dilator 25 and a guiding pin or mandril 26.

The distal end of the dilator 25 is preferably formed as a tapered end and the inner diameter of the dilator 25 matches the diameter of the guide wire 24. The dilator 25 may include a radiopaque section 20. The position of the radiopaque section 20 on the dilator 25 is selected such that it enables a physician to ensure under fluoroscopy that the dilator 25 has been inserted into the collecting system to a certain degree.

Although fluoroscopy is one method of visualizing medical devices such as the dilator 25 and the catheter 1, ultrasound techniques may also be used for visualization of such medical devices within a body cavity. Ultrasound techniques for visualizing these devices within body cavities are well known to those of skill in the art.

The tubular body 2, the first connector piece 3, the slidable member 4 and the dilator 25 are fabricated from commonly used medical grade plastics using standard techniques. For example, the plastic can be one or more of polyurethane, polypropylene, polyethylene, nylon, polyethylene terephthalate, polyethen, Hd-polyethen, latex, and any other suitable polymer, as well as a Pebax® material. Furthermore, the tubular body 2 can be fabricated of more than one material. Materials having different stiffness can be used to produce a tubular body 2 having different stiffness in different regions. Materials having different stiffness can be welded together or unified through melting. Alternatively, a process can be used wherein the stiffness of the material can be regulated during injection moulding of the tubular body 2. If desired, the tubular body 2 can be produced having different thickness in different sections. A memory metal or a spring metal can also be used in the tubular body 2.

The guiding pin or mandril 26 can be made from any medical grade metal or polymer. The metal can be, for example, stainless steel, nitinol, or titanium. The polymer can be one or more of the polymers described above. The elongate member 5 can be made of, for example, nylon.

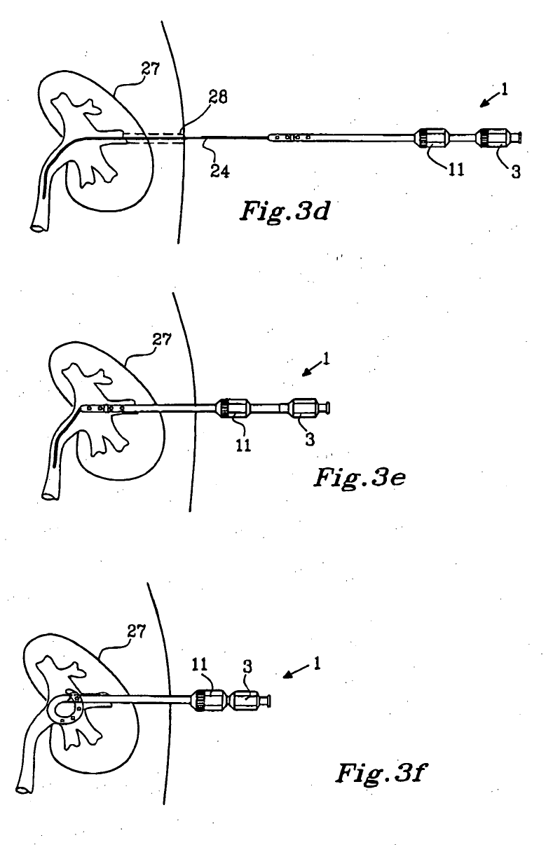

A procedure of inserting the catheter 1 according to the first embodiment into the renal pelvis for the purpose of draining the renal pelvis and securing the catheter 1 in place by utilization of all of the medical devices in the set of medical devices 22 will now be explained with reference to the FIGS. 3a-f, in which the procedure is schematically shown. Initially, the hollow needle 23 is used to percutaneously puncture the kidney 27 to provide an access path 28 to the collecting system 29, such as the renal pelvis. The penetration is done such that the needle 23 enters into a calyx 30 (FIG. 3a). The guide wire 24 is then passed through the lumen of the hollow needle 23 such that it extends into the collecting system (FIG. 3b). With the guide wire 24 in position within the body cavity, the needle 23 is then withdrawn. The dilator 25 is thereafter passed over the guide wire 24 and inserted into the kidney tissue (FIG. 3c). Passing the dilator 25 into the tissue widens or dilates the access path 28 created Initially by the needle 23. A physician verifies optically under fluoroscopy, ultrasound or x-ray that the radiopaque section 20 is positioned within the collecting system. Then the dilator 25 is withdrawn, which leaves a dilated access path 28.

The catheter 1 is next introduced over the guide wire 24 (FIG. 3d). In order to ease the introduction of the catheter 1 into the dilated access path 28, the rigid guiding pin or mandril 26 (not shown) may first be inserted into the catheter 1 to provide longitudinal rigidity and support to the catheter 1. In this manner, the catheter 1 has less of a tendency to get stuck during insertion, which otherwise might occur due to flexibility of the catheter 1. Generally, the catheter 1 has to be smooth and bend easily without collapsing.

A physician verifies optically under fluoroscopy, ultrasound or x-ray that the radiopaque section 20 of the catheter 1 is positioned within the collecting system. In order to enable the loop formation in a body cavity, the second openings 16 have to be inserted into the body cavity. Thus, the radiopaque section 20 is preferably positioned on the tubular body 2 such that it may be ensured that the second openings 16 are positioned within the body cavity to be drained. The physician may then ensure that the elongate member 5 is stretched or taut by pushing the second connector piece 11 to a certain position (FIG. 3e). The guide wire 24 is removed before the loop formation starts. When the second connector piece 11 is held steady at the position at which the elongate member 5 is stretched and the tubular body 2 is advanced such that the first connector piece 3 is moved in the direction of the second connector piece 11, a loop starts to form in the distal region 6 of the tubular body 2. This is due to that the elongate member 5 is stretched and cannot be elongated due to its lack of elasticity, whereby the first openings 15 can not be further advanced into the body cavity when the first connector piece 3 is advanced in the direction of the second connector piece 11. As described above, in order to obtain a complete loop, the tubular body 2 is advanced until the first connector piece 3 and the second connector piece 11 are adjacent to each other. At this point a loop is formed in the distal region 6 of the tubular body 2 (FIG. 3f) and the first connector piece 3 and the second connector piece 11 may be coupled together, e.g. by a thread engagement, a bayonet type lock or by any other suitable locking means. The second connector piece 4 has to be held steady outside the body, or at least essentially steady, during the formation of the loop in order to keep the elongate member 5 stretched. In this way, a loop is easily formed and the catheter 1 is locked into position within the collecting system (i.e. renal pelvis) with the loop effectively anchoring the catheter 1 within the kidney. Furthermore, the locking of the second connector piece 11 to the first connector piece 3 ensures that the loop is kept during a drainage procedure. Optionally, the catheter 1 may be secured to the patient's skin during a drainage procedure. Since the second connector piece 11 is kept outside the body, the tubular body 2 and small parts of the elongate member 5 are the only artificial devices left in the body during a drainage procedure. This is advantageous during certain medical conditions. During the formation of the loop, the guiding pin 26, if used, should be removed to prevent it from interfering with the loop. Alternatively, the guiding pin 26 can be kept in place but it should not be advanced together with the catheter 1. After finished drainage, the lock, if any, between the first connector piece 3 and the second connector piece 11 is released. Then the tubular body 2 is withdrawn, whereby the loop reduces its size and finally is eliminated.

The procedure of inserting and securing the catheter 1 in place can be summarized as follows: (1) use a needle 23 to create a channel through tissue to the cavity to be drained; (2) insert a guide wire 24 through the needle 23 and advance the guide wire 24 into the cavity; (3) remove the needle 23 and pass a dilator 25 over the guide wire 24 to dilate the tissue; (4) remove the dilator 25 and pass a catheter 1 over the guide wire 24; (5) optically verify that the distal region 6 of the tubular body 2 is correctly positioned within the body cavity; (6) ascertain that the elongate member 5 is stretched and keep the elongate member 5 stretched and remove the guide wire 24; (7) advance the first connector piece 3 in direction of the second connector piece 11 to form a loop at the distal region 6 of the tubular body 2; (8) couple the first connector piece 3 and the second connector piece 11 to each other after a loop of a desired size has been formed; and, optionally, (9) secure the catheter 1 to the patient's skin.

By the above described method, the catheter 1 is inserted into a body cavity such that the second openings 16 are positioned within the body cavity and the loop is thereafter formed by advancing the second openings 16 further into the body cavity. Thus, the loop is then not formed at an entry point into a body cavity. However, for providing a loop at an entry point into a body cavity, the catheter 1 is inserted into the body cavity such that the second openings 16 are positioned in the body cavity at the entry point. The loop is then formed by moving the slidable second connector piece 11 in the direction of the first connector piece 3 instead of holding the second connector piece 11 steady and advancing the tubular body 2 further into the body cavity by moving the first connector piece 3 in the direction of the second connector piece 11 as described above. Thereby, the tubular body 2 is not further advanced into the body cavity during the formation of the loop, but the loop is formed by pulling the first openings 15 in the direction of the second openings 16.

Furthermore, a procedure of inserting the catheter 1 according to the first embodiment was above described in accordance with one common type of method for inserting a drainage catheter into a body cavity. However, any other suitable method known in the art may also be utilized for insertion of the catheter 1 according to the invention into a body cavity.

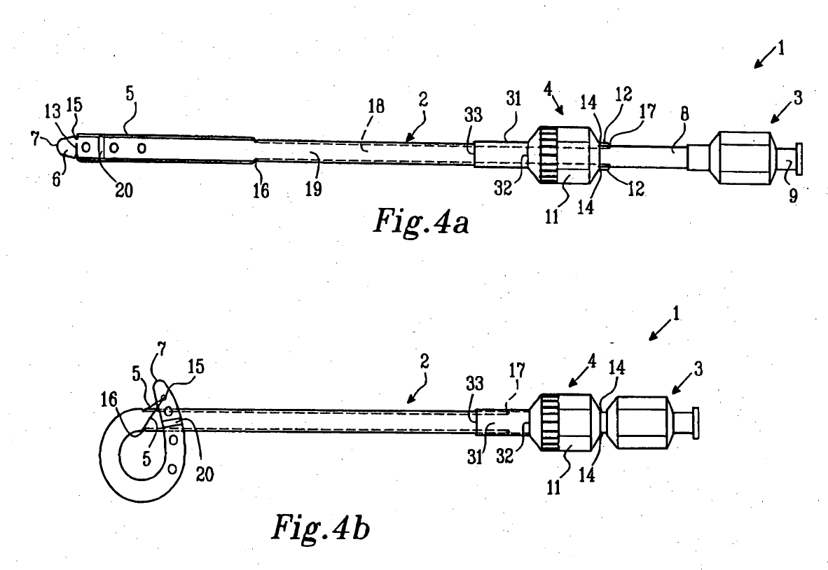

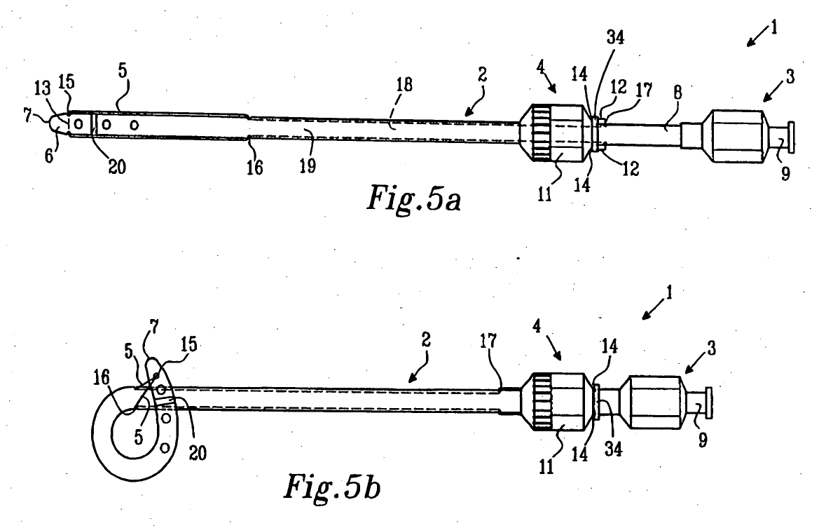

A second embodiment of the catheter 1 according to the invention differs from the first embodiment only in that the one slidable member 4 further comprises a sheath 31 having a longitudinal channel passing between a proximal end 32 and a distal end 33. The sheath 31 is joined to the second connector piece 11 at the proximal end 32. Thus, the slidable member 4 consists of the second connector piece 11 and the sheath 31 in the second embodiment. Both the second connector piece 11 and the sheath 31 are arranged to be positioned outside the body. The sheath 31 is arranged to encircle at least a portion of the circumference of the tubular body 2. The length of the sheath 31 may vary, but it is adapted such that the distal end 33 of the sheath 31 is arranged to be positioned outside the body. A catheter 1 according to the second embodiment is shown in FIGS. 4a and 4b. In the second embodiment, the proximal attachment parts 12 of the elongate member 5 need not be attached to the second connector piece 11, but may instead be attached to the sheath 31. The proximal attachment points 14 may be positioned anywhere on the second connector piece 11 or the sheath 31.

In use, the second embodiment of the catheter 1 functions mainly in the same way as the first embodiment of the catheter 1, whereby the sheath 31 is positioned outside the body. A total length of the slidable member 4 may be set such that the third openings 17 is covered by the sheath 31 (FIG. 4b), or the second connector piece 11, when a loop has been formed in the distal region 6 of the tubular member 2. Thereby, leakage of drainage liquid through the third openings 17 is prevented.

In a variant of the second embodiment (not shown) a stop member may be joined to the distal end 33 of the sheath 31 in order to prevent the sheath 31 from entering into the body. For example, such a stop member may be configured as a plate-like member.

FIGS. 5a-b show a third embodiment of the catheter 1 according to the invention, which is a variant of the first embodiment. The third embodiment differs from the first embodiment in that the catheter 1 comprises a further slidable member 4, i.e. it comprises two separate slidable members 4, whereby one slidable member 4 consists of the second connector piece 11 and one slidable member 4 is a ring member 34 (i.e. pulling means, stop or stopping device). The ring member 34 is arranged to be positioned between the second connector piece 11 and the first connector piece 3. In the third embodiment, the elongate member 5 is attached to the ring member 34 at its two proximal attachment parts 12 instead of to the second connector piece 11 as in the first embodiment. The second connector piece 11 may or may not be separable from the tubular body 2. The same applies to the ring member 34.

The ring member 34 is arranged to encircle at least a portion of the tubular body 2 and is slidable along the tubular body 2. The ring member 34 is slidable along the tubular body 2 independently of the second connector piece 11. The elongate member 5 may be stretched by letting the ring member 34 abut against the second connector piece 11 and placing the second connector piece 11 in such a position that the elongate member 5 is stretched.

The interaction between the ring member 34 and the second connector piece 11 is in the third embodiment the key to forming the loop in the distal region 6 of the tubular body 2. In order to form a loop in the distal region 6 of the tubular body 2, the second connector piece 11 may initially be pushed to such a position along the tubular body 2 that the ring member 34 abuts against the second connector piece 11 and such that the elongate member 5 is stretched (FIG. 5a). Holding the second connector piece 11 steady at that position and advancing the first connector piece 3 in the direction of the second connector piece 11 thereafter implies that a loop is formed in the distal region 6 of the tubular body 2 due to that the elongate member 5 already is stretched and cannot be elongated due to its lack of elasticity (FIG. 5b). The formation of the loop of the catheter 1 according to the third embodiment occurs in accordance with the formation of the loop of the catheter 1 according to the first embodiment described above. Consequently, as mentioned above, a loop may also be formed by moving the second connector piece 11 in the direction of the first connector piece 3 instead of advancing the tubular body 2 by moving the first connector piece 3 in the direction of the second connector piece 11. Then the second connector piece 11 pushes the ring member 34 in the direction of the first connector piece 3, whereby the distal coupling part 13 and the first openings 15 are moved in the direction of the second openings 16.

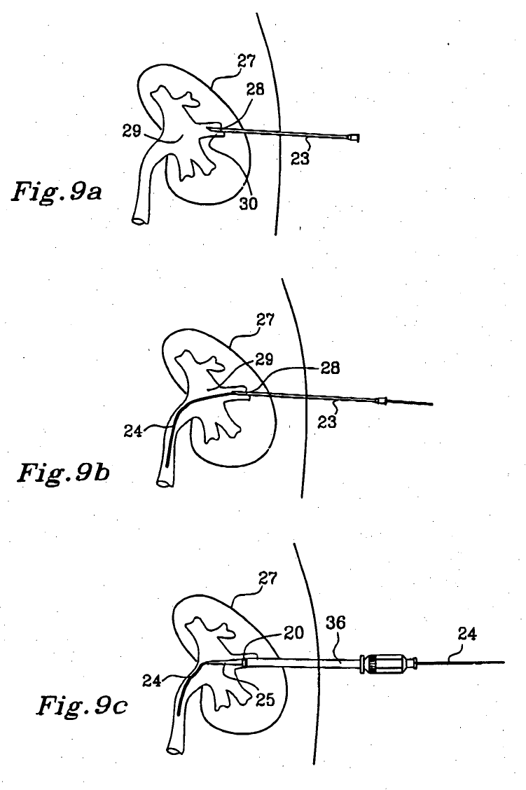

FIG. 6 shows a fourth embodiment of the catheter 1 according to the present invention, which is a variant of the third embodiment. The fourth embodiment differs from the third embodiment only in that a sheath 31 is joined to the second connector piece 11 in accordance with the second embodiment. Thus, a catheter 1 according to the fourth embodiment comprises two separate slidable members 4, whereby one slidable member 4 consists of the second connector piece 11 and the sheath 31 having a longitudinal channel passing between the proximal end 32 and the distal end 33. The sheath 31 is joined to the second connector piece 11 at the proximal end 32. The other slidable member 4 is a ring member 34, which is arranged to be positioned between the second connector piece 11 and the first connector piece 3. In the fourth embodiment, the elongate member 5 is attached to the ring member 34 with its two proximal attachment parts 12. In accordance with the third embodiment, the interaction of the ring member 34 and the second connector piece 11 is the key to forming the loop when a catheter 1 according to the fourth embodiment is used.

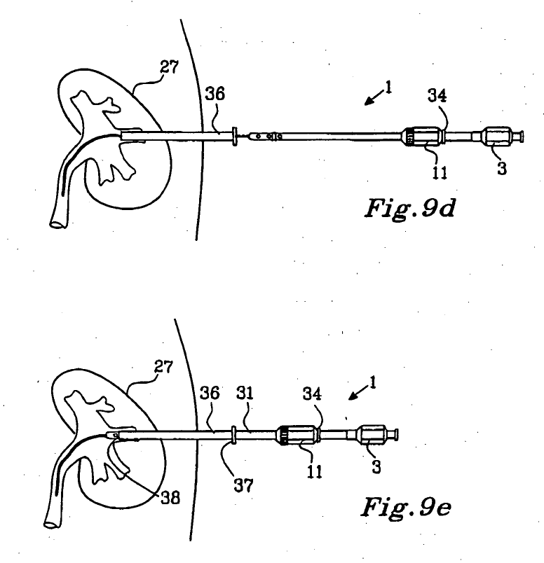

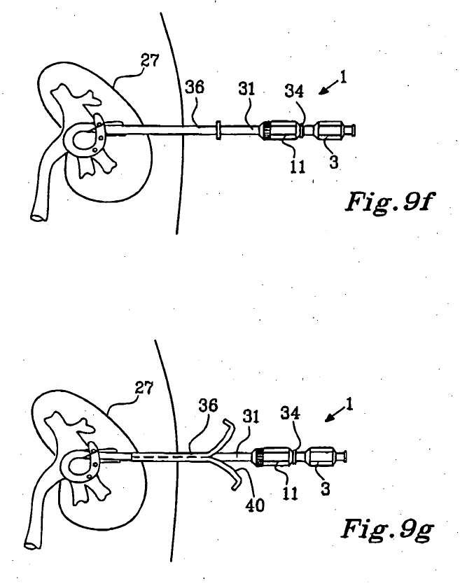

In variants of any of the above mentioned embodiments, the second openings 16 and the third openings 17 are excluded from the tubular body 2. Then the elongate member 5 passes between the proximal attachment points 14 and the first openings 15 outside the tubular body 2. However, in such a variant, an attachment member 41 is comprised instead of the second openings 16 for anchoring the elongate member 5 at a position along the tubular body 2 such that the loop may be formed. For example, the attachment member 41 is a ring, a ring member, a slot on the outer surface 19 of the tubular body 2 or any other suitable means. One such variant, which is a variant of the third embodiment, is shown in FIG. 7a. The catheter 1 shown in FIG. 7a comprises two slidable members 4, whereby one slidable member 4 is constituted by the second connector piece 11 and one slidable member 4 is constituted by the ring member 34. However, the second connector piece 11 is shown separated from the tubular body 2 in FIG. 7a.

Referring to FIG. 7a, starting from one of the proximal attachment points 14 at the ring member 34, the elongate member 5 runs along the outer surface 19 of the tubular body 2 to one first opening 15 in the distal region 6 of the tubular body 2 (or to a point near the distal end 7), where it penetrates the tubular body 2 and exits through the other first opening 15. Then it runs back along the outer surface 19 of the tubular body 2 and attaches to the ring member 34 at the other proximal attachment point 14. A ring member 41 is provided around the tubular body 2 and encloses the elongate member 5 for anchoring the elongate member 5 at a position along the tubular body 2.

FIG. 7b shows a fifth embodiment of the catheter 1, which is a variant of the first embodiment. The fifth embodiment differs from the first embodiment in that the second connector piece 11, i.e. the one slidable member 4 that is constituted by the second connector piece 11, is arranged to be positioned proximal to the first connector piece 3 instead of distal to the first connector piece 3. However, the first connector piece 3 is still arranged to be positioned outside the body. Furthermore, the fifth embodiment of the catheter 1 differs from the third embodiment in that the tubular body 2 only comprises two pairs of openings; the two first openings 15 and the two second openings 16. Thus, the two third openings 17 are excluded in the fifth embodiment. The distal coupling part 13 of the elongate member 5 is coupled to the distal region 6 of the tubular body 2 by passing through the two first openings 15. From the first openings 15, the elongate member 5 passes on the outer surface 19 of the tubular body 2 to the second openings 16. From the second openings 16, the elongate member 5 passes inside the inner lumen 18 of the tubular body 2 to the proximal end 9 of the tubular body 2 instead of to the third openings 17 as in the first embodiment. At the proximal end 9, which is an open end, the elongate member 5 passes out to the outside of the tubular body 2 and is attached with its proximal attachment parts 12 at the proximal attachment points 14 on the second connector piece 11. The attachments of the elongate member 5 to the second connector piece 11 may be provided anywhere on the second connector piece 11. In use of the fifth embodiment, the elongate member 5 is stretched when the second connector piece 11 is held at a certain position along the tubular body 2. Further advancing the second connector piece 11 in the direction of the first connector piece 3 creates a loop in the distal region 6 of the tubular body 2 due to that the elongate member 5 is stretched and can not be further elongated due to its lack of elasticity. When the second connector piece 11 in the fifth embodiment is moveable in the direction of the first connector piece 3, the elongate member 5 is pulled between the proximal end 9 and the proximal attachment points 14 outside the tubular body 2, whereby a loop forms in the distal region 6 due to that the first openings 15 are moved in the direction of the second openings 16. The second connector piece 11 may be moved in the direction of the first connector piece 3 until they mate. The second connector piece 11 and the first connector piece 3 may then be coupled together, e.g., by a thread engagement, a bayonet type lock or by any other suitable locking means, such that the loop is prevented from reducing its size or being eliminated during a drainage procedure. In the fifth embodiment, the first connector piece 3 works as a stop or stopping device due to that the second connector piece 11 is prevented from further advancement after mating with the permanently attached first connector piece 3. In use of the fifth embodiment, the tubular body 2 has to be inserted into a body cavity to such a degree that the second openings 16 is positioned within the body cavity.

In a sixth embodiment (not shown), which is a variant of the fifth embodiment, the tubular body 2 comprises further the third openings 17. The third openings 17 are in the sixth embodiment positioned proximal to the first connector piece 3. The first openings 17 may in the sixth embodiment be positioned on the tubular body 2 anywhere between the permanently attached first connector piece 3 and the proximal end 9 of the tubular body 2. The distal coupling part 13 of the elongate member 5 is coupled to the distal region 6 of the tubular body 2 by passing through the first openings 15. From the first openings 15, the elongate member 5 passes on the outer surface 19 of the tubular body 2 to the second openings 16. Then the elongate member 5 passes from the second openings 16 to the third openings 17 along the inner lumen 18. At the third openings 17, the elongate member 5 passes out to the outside of the tubular body 2 and passes outside the tubular body 2 to the attachment points 14 on the second connector piece 11. The attachments of the elongate member 5 to the second connector piece 11 may be provided anywhere on the second connector piece 11. In use of the sixth embodiment, the elongate member 5 is stretched when the second connector piece 11 is held at a certain position along the tubular body 2. Further advancing the second connector piece 11 in the direction of the first connector piece 3 creates a loop in the distal region 6 of the tubular body 2 due to that the elongate member 5 is stretched and can not be further elongated due to its lack of elasticity. Thereby, a loop forms in the distal region 6 due to that the first openings 15 are moved in the direction of the second openings 16. When the second connector piece 11 in the sixth embodiment is moved in the direction of the first connector piece 3, the elongate member 5 is pulled between the third openings 17 and the proximal attachment points 14 on the outside of the tubular body 2. In use of the sixth embodiment, the tubular body 2 has to be inserted into a body cavity to such a degree that the second openings 16 are positioned within the body cavity.

Furthermore, variants (not shown) of any of the first, second, third, fourth or sixth embodiments may comprise only one third opening 17 instead of two third openings 17. Then the elongate member 5 passes through the one third opening 17 two times instead of passing through two third openings 17.

In addition, further variants (not shown) of any of the first, second, third, fourth or sixth embodiments may comprise only one second opening 16 instead of two second openings 16. Then the elongate member 5 passes through the one second opening 16 two times instead of passing through two second openings 16.

Still further variants (not shown) of any of the first, second, third, fourth or sixth embodiments may comprise only one second opening 16 instead of two second openings 16 and only one third opening 17 instead of two third openings 17. Then the elongate member 5 passes through the one second opening 16 two times instead of through two second openings 16 and through the one third opening 17 two times instead of through two third openings 17.

A variant (not shown) of the fifth embodiment may comprise one second opening 16 instead of two second openings 16, whereby the elongate member 5 passes through the one second opening 16 two times instead of through two second openings 16.

Furthermore, in further variants (not shown) of any of the above described embodiments, the catheter 1 comprises two elongate members 5 instead of one elongate member 5, each having one proximal attachment part 12 and one distal coupling part 13. The distal coupling part 13 of each of the two elongate members 5 is attached to the outer surface 19 of the tubular body 2 in the distal region 6 of the tubular body 2. The attachment may be provided by a knot, by gluing, a metal band or molding etc, which then may replace the first openings 15 in the above described embodiments. The two elongate members 5 replace then the one elongate member 5 in the above described embodiments and it may be seen as the one elongate member 5 in the above described embodiments is divided into two parts. Each elongate member 5 extends then as a single length of a thread between the coupling of the distal coupling part 13 to the distal region 6 of the tubular body 2 and the proximal attachment point 14. The proximal attachment part 12 and the distal coupling part 13 may then each constitute an end of the elongate member 5. Furthermore, such a variant may comprise one or two second openings 16 and one or two third openings 17 in accordance with any of the above described embodiments, whereby the two elongate members 5 may pass through the same or different second openings 16 and the same or different third openings 17. If it is a variant of the fifth embodiment, it does not comprise any third opening 17. Furthermore, the two elongate members 5 may also extend completely outside the tubular body 2.

In other variants (not shown) of any of the above described embodiments, the catheter 1 comprises one elongate member 5 having one proximal attachment part 12 and one distal coupling part 13. The distal coupling part 13 of the elongate members 5 is attached to the outer surface 19 of the tubular body 2 in the distal region 6 of the tubular body 2. The attachment may be provided by a knot, by gluing, a metal band or molding etc, which then may replace the first openings 15 in the above described embodiments. Thus, the elongate member 5 extends as a single length of a thread between the coupling of the distal coupling part 13 to the distal region of the tubular body 2 and the proximal attachment point 14. However, the coupling of the distal coupling part 13 to the distal region 6 may comprise the elongate member 5 passing through the tubular body 2 through the first openings 15 and being attached by a knot, by gluing, a metal band or molding on the other side of the tubular body 2. Furthermore, the elongate member 5 in such a variant may either extend only outside the tubular body 2 or both along the outer surface 19 and the inner lumen 18 of the tubular body 2 between the coupling of the distal coupling part 13 to the distal region 6 of the tubular body 2 and the proximal attachment point 14.

If such a variant, which comprises one elongate member 5 with one proximal attachment part 12, is a variant of any of the first, second, third, fourth or sixth embodiments, the tubular body 2 comprises one second opening 16 instead of two second openings 16 and one third openings 17 instead of two third openings 17. Thus, then the elongate member 5 passes from the coupling to the distal region 6 to the second opening 16 along the outer surface 19 of the tubular body 2. Between the second opening 16 and the third opening 17, the elongate member 5 passes inside the inner lumen 18. At the third opening 17 the elongate member 5 passes out to the outside of the tubular body 2 and to the proximal attachment point 14.

If such a variant, which comprises one elongate member 5 with one proximal attachment part 12, is a variant of the fifth embodiment, the tubular body 2 comprises one second opening 16 instead of two second openings 16.

If the elongate member(s) 5 in any of the above described embodiments is/are coupled to the distal region 6 at a set distance from the distal end 7 of the tubular body 2, the tubular body 2 extends beyond the loop by the set distance when the loop is formed. Furthermore, if the elongate member(s) 5 is/are coupled to the distal region 6 at such a set distance, drainage openings 10 may be provided at the distal end 7 or at the position of the coupling of the elongate member 5 to the distal region 6 or at both those positions.

For example, if it is desirable to drain the kidney (collecting system) at a point further down in the kidney, e.g., closer to the ureter, it is necessary to provide a relatively long tip that reaches as much as up to approximately 10 cm beyond the point of entry into the collecting system. Nonetheless, it is advantageous to position the loop at that entry point into the collecting system to provide a reliable fixation.

In still other variants of the above mentioned embodiments, the catheter is configured to drain two body cavities (not shown). In such a variant, the tubular body comprises two sets of drainage openings. One set of drainage openings is then, for example, positioned at the distal end of the tubular body and one set of drainage openings is then positioned in a middle section of the tubular body. Furthermore, the elongate member(s) is/are coupled by its distal coupling part(s) 13 to the tubular body in the middle section of the tubular body, whereby a loop may be formed at the middle section in the above described manner by stretching of the elongate member and coupling of the first connector piece and the second connector piece together. Thereby, the catheter may be secured at an entry point into a body cavity and the body cavity may both be drained at the entry point and at the position where the drainage openings at the distal end of the catheter are located. However, a further loop may also be provided in such a variant at the distal end of the tubular body. The further loop may be formed using standard methods, e.g., placing the catheter over a curved mandril and placing the assembly in a heat oven to impart the curve in the mandril to the catheter. Thus, such a loop is formed before the catheter is inserted into a body cavity. However, such a loop is preferably stretched before introduction into a body cavity. Such a variant of the catheter according to the invention, may, for example, be used to drain the kidney and the bladder.

The catheterization kit 21 according to the invention may comprise any of the above mentioned embodiments of the catheter 1.

Another catheterization kit 35 according to the invention comprises the catheter 1 of any of the above described embodiments, in which the second connector piece 11 is positioned distal to the first connector piece 3, and a split-away sheath 36. One example of such a kit 35 is shown in FIGS. 8a and 8b, which kit 35 comprises the catheter 1 according to the fourth embodiment. Thus, the catheter 1 shown in FIGS. 8a and 8b comprises two slidable members 4, whereby one slidable member 4 is constituted by the second connector piece 11 and the sheath 31 and one slidable member 4 is a ring member 34.

The split-away sheath 36 is arranged to encircle at least a portion of the circumference of the tubular body 2 and has a longitudinal channel passing between a proximal end 37 and a distal end 38. The proximal end 37 is arranged to be positioned outside the body and the distal end 38 is arranged to be positioned inside the body. The split-away sheath 36 is intended to be utilized for guiding and protecting the catheter 1 during the insertion of the catheter 1 into a body cavity, but to be removed after insertion. The split-away sheath 36 may be removed before or after the loop formation. Preferably, the split-away sheath 36 comprises also a stop or stop member 39 at its proximal end 37, which stop member 39 prevents the proximal end 37 of the split-away sheath 36 from being inserted into the body.

The catheter 1 may be insertable into the split-away sheath 36 with the distal end 7 first. The slidable member 4 comprising the second connector piece 11 may abut the proximal end 37 of the split-away sheath 36. When the catheter 1 has been inserted into the split-away sheath 36 to a certain degree, the slidable member 4 comprising the second connector piece 11 abuts the proximal end 37 of the split-away sheath 30, the ring member 34, if any, abuts the second connector piece 11 and the elongate member 5 is stretched. The length of the split-away sheath 36 may be adjusted such that the coupling of the elongate member 5 to the distal region 6 is positioned just outside the distal end 38 of the split-away sheath 36 when the elongate member 5 is stretched. However, the split-away sheath 36 may also be shorter than that length, whereby the coupling of the elongate member 5 to the distal region 6 extends beyond the distal end 38 of the split-away sheath 36 by a set distance when the elongate member 5 is stretched. For example, the length of the split-away sheath 36 may be adjusted such that the second opening(s) 16 is/are positioned just outside the distal end 38 of the split-away sheath 36 when the elongate member 5 is strectched. Furthermore, the length of the elongate member 5 may of course also be adjusted to the length of the split-away sheath 36.

In the embodiment shown in FIGS. 8a and 8b, the length of the split-away sheath 36 and the length of the elongate member 5 are adjusted to each other such that the coupling of the elongate member 5 to the distal region 6 is positioned just outside the distal end 38 of the split-away sheath 36 when the elongate member 5 is stretched i.e. such that the first openings 15 are positioned just outside the distal end 38 of the split-away sheath 36 when the elongate member 5 is stretched. Then the distal end 33 of the sheath 31 abuts the proximal end 37 of the split-away sheath 36 and the ring member 34 abuts the second connector piece 11 (fig. Bb). Further advancement thereafter of the first connector piece 3 in the direction of the second connector piece 11 implies that the tubular body 2 begins to be expelled out of the split-away sheath 36. Since the elongate member 5 is non-elastic, it remains a general constant length and the coupling of the elongate member 5 to the distal region 6 of the tubular body 2 is therefore kept adjacent to the distal end 38 of the spilt-away sheath 36 during such an advancement, whereby a loop is formed at the distal end 38 of the split-away sheath 36. The use of the catheterization kit 35 shown in FIGS. 8a and 8b in a body cavity will be further described below.

The split-away sheath 36 comprises two longitudinal separation lines 40 extending from the proximal end 37 to the distal end 38 of the split-away sheath 36, whereby the split-away sheath 36 easily may be divided into two parts and removed from the tubular body 2. The separation line 40 may, for example, be a perforation or may be provided by a thinning of the material. Furthermore, a kerf, such as a notch or a groove, may be provided in the stop member 39 extending from the proximal end 37 to each separation line 40 or may constitute a part of each separation line 40. The use of the separation lines 40 will be further described below.

Furthermore, the split-away sheath 36 may comprise a radiopaque section 20 and the position of the radiopaque section 20 on the split-away sheath 36 is set such that a physician may utilize the radiopaque section 20 for ensuring that the distal end 38 is inserted into a body cavity. Preferably, the radiopaque section 20 is positioned near or at the distal end 38.

The catheterization kit 35 may further comprise one, several or all of the medical devices in the set of medical devices 22 shown in FIG. 2.

A procedure of using a catheterization kit 35 comprising the catheter 1 according to the fourth embodiment as well as all of the medical devices in the set of medical devices 22 will now be described with reference to FIGS. 9a-g, in which the procedure is schematically shown. The procedure comprises inserting the catheter 1 into the renal pelvis for the purpose of draining the renal pelvis and securing the catheter 1 in place. Initially, the hollow needle 23 is used to percutaneously puncture the kidney 27 to provide an access path 28 to the collecting system 29, such as the renal pelvis. The penetration is done such that the needle 23 enters into a calyx 30 (FIG. 9a). The guide wire 24 is then passed through the lumen of the hollow needle 23 such that it extends into the collecting system (FIG. 9b). With the guide wire 24 in position within the body cavity, the needle 23 is then withdrawn. The dilator 25 is thereafter inserted into the split-away sheath 36 and the assembly of the dilator 25 and the split-away sheet 36 is passed over the guide wire 24 and inserted into the kidney tissue. The dilator 25 comprises a radiopaque section 20. The length of the dilator 25 and the position of the radiopaque section 20 on the dilator 25 are set such that the radiopaque section 20 is located just at the distal end 38 of the split-away sheath 36 when the dilator 25 is completely inserted into the split-away sheath 36. Thereby, it may be ascertained when the split-away sheath 36 has been positioned just inside the collecting system by looking for the radiopaque section 20 under fluoroscopy (FIG. 9c). Alternatively, a radiopaque section 20 may be provided on the spilt-away sheath 36.

Passing the assembly of the dilator 25 and the split-away sheath 36 into the tissue widens or dilates the channel created initially by the needle 23. To dilate the tissue in a manner that is as atraumatic as possible, there should be a smooth transition between the split-away sheath 36 and the extension of the dilator 25 out from the split-away sheath 36. For example, the distal tip of the split-away sheath 36 can be tapered to have an inner diameter that is minimally larger than the outer diameter of the dilator 25. This is most easily achieved by tapering the distal end 38 of the split-away sheath 36 to a fairly small angle, such as less than approximately 45°, preferably less than 30°, most preferably less than 20°. In general, the smaller the angle the better and only manufacturing techniques limit the angle value. Of course, if the split-away sheath 36 is made from a material that is thin enough, a taper may not be needed at all if there is a very close fit of the dilator 25 within the split-away sheath 36. To decrease the friction between the split-away sheath 36 and the dilator 25 a hydrofil friction coating may be applied on the dilator 25. The dilator 25 may be connected to the proximal end 37 of the split-away sheath 36 through a hub at the proximal end of the dilator 25.

The dilator 25 is next withdrawn, which leaves the split-away sheath 36 in position within the collecting system. The catheter 1 may thereafter be introduced into the split-away sheath 36 (FIG. 9d). To ease the introduction of the catheter 1 into the spilt-away sheath 36, the rigid guiding pin 26 (not shown) may first be inserted into the catheter 1 to provide longitudinal rigidity and support to the catheter 1. In this manner, the catheter 1 has less of a tendency to get stuck in the split-away sheath 36, which otherwise might occur due to the flexibility of the catheter 1.

To decrease the friction between the split-away sheath 36 and the catheter 1 a friction reducing coating may be applied on the tubular body 2 of the catheter 1.

Generally, the split-away sheath 36 as well as the assembly of the split-away sheath 36 and the catheter 1 has to be smooth and bend easily without collapsing.

The elongate member 5 will be tight or stretched when the catheter 1 has been inserted into the split-away sheath 36 to such a degree that the coupling of the elongate member 5 to the distal region 6 is positioned just outside the distal end 38 of the split-away sheath 36. Thereby the sheath 31 abuts the proximal end 37 of the split-away sheath 36 and the ring member 34 abuts the second connector piece 11 (FIG. 9e). The elongate member 5 is stretched because the length of the split-away sheath 36 and the length of the elongate member 5 are adjusted to each other for providing the stretch. Because the elongate member 5 is stretched and cannot be elongated due to its lack of elasticity, and because the coupling of the elongate member 5 to the distal region 6 cannot move further away from the distal end 38 of the split-away sheath 36, advancing the catheter 1 further into the split-away sheath 36 creates a loop in the tubular body 2. During the formation of the loop, the guiding pin 26, if any is used, should be removed to prevent it from interfering with the loop. Alternatively, the guiding pin 26 can be kept in place but it should not be advanced together with the catheter 1.

As a consequence of advancing the catheter 1 further into the split-away sheath 36, the tubular body 2 will therefore begin to be expelled out from the split-away sheath 36, whereby the loop will start to form at the distal end 38 of the split-away sheath 36. The physician advances the catheter 1 until the first connector piece 3 of the catheter 1 mates with the second connector piece 11, whereby the ring member 34 is positioned between the first connector piece 3 and the second connector piece 11. At this position the first connector piece 3 and the second connector piece 11, respectively, can be coupled together, e.g., by a thread engagement, a bayonet type lock or by any other suitable locking means, such that the loop is prevented from reducing its size or being eliminated during a drainage procedure. In this manner, the catheter 1 is locked into position within the collecting system (i.e. renal pelvis) with the loop effectively anchoring the catheter within the kidney (FIG. 9f).

Thereafter the split-away sheath 36 may be removed by dividing the split-away sheath 36 along the two longitudinal separation lines 40 and draw out the two divided parts (FIG. 9g). Optionally, the catheter 1 may then be secured to the patient's skin. Then the only artificial devices left in the body during the drainage procedure are the tubular body 2 and small parts of the elongate member 5. After finished drainage, the lock, if any, between the first connector piece 3 and the second connector piece 11 is released. Then the tubular body 2 is withdrawn, whereby the loop reduces its size and finally is eliminated.

Furthermore, a procedure of using one embodiment of the catheterization kit 35 was above described in accordance with one common type of method for inserting a drainage catheter into a body cavity. However, any other suitable method known in the art may also be utilized when a catheterization kit 35 according to the invention is utilized.

Although the above described embodiments of the catheter according to the invention have been described with reference to application in catheterization of the renal pelvis, it is to be understood that the catheter according to the invention have broader application than use only in the renal pelvis. For example, the catheter according to the invention will function equally well for draining other body cavities of a human or an animal, such as but not limited to the urinary bladder, the gall bladder, abscesses, peritoneal and thoracic cavity. Accordingly, other embodiments are within the scope of the following claims.

Thus, while there have been shown and described and pointed out fundamental novel features of the invention as applied to preferred embodiments thereof, it will be understood that various omissions and substitutions and changes in the form and details of the devices illustrated, and in their operation, may be made by those skilled in the art without departing from the spirit of the invention. For example, the tubular body could comprise more openings through which the elongate member may pass than the number of openings described above. Thus, the elongate member may be configured to run between a proximal attachment part and the coupling to the distal region of the tubular body in another way than any of those described above. It is expressly intended that all combinations of those elements which perform substantially the same function in substantially the same way to achieve the same results are within the scope of the invention. Moreover, it should be recognized that structures and/or elements shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A catheter comprising:
a tubular body having a distal region and a proximal region;
a first connector piece being permanently attached to the tubular body and being arranged to be positioned outside the body;
at least one member being arranged to encircle at least a portion of the circumference of the tubular body and to be slidable along the tubular body, whereby each of the at least one slidable member is arranged to be positioned outside the body and one of the at least one slidable member comprises a second connector piece connectable to the first connector piece, and
at least one elongate member, each of which having at least one proximal attachment part and a distal coupling part, the distal coupling part of each elongate member being coupled to the distal region of the tubular body and each of the at least one proximal attachment part being attached to one of the at least one slidable member at a proximal attachment point, whereby each elongate member extends from each respective proximal attachment point to the distal coupling part, whereby the slidable member to which each of the at least one proximal attachment part is attached is slidable to a position adjacent the first connector piece.

2. The catheter according to claim 1, wherein the catheter comprises one slidable member and wherein the slidable member consists of the second connector piece.

3. The catheter according to claim 2, wherein the second connector piece is arranged to be positioned distal to the first connector piece.

4. The catheter according to claim 2, wherein the second connector piece is arranged to be positioned proximal to the first connector piece.

5. The catheter according to claim 1, wherein the catheter comprises one slidable member and wherein the slidable member consists of the second connector piece and a sheath having a longitudinal channel passing between a proximal end and a distal end, the sheath being joined to the second connector piece at the proximal end and the second connector piece being arranged to be positioned distal to the first connector piece.

6. The catheter according to claim 1, wherein the catheter comprises two slidable members, whereby one slidable member consists of the second connector piece and one slidable member is a ring member, whereby each at least one proximal attachment part of each of the at least one elongate member is attached to the ring member, whereby the ring member is arranged to be positioned between the second connector piece and the first connector piece and whereby the second connector piece is arranged to be positioned distal to the first connector piece.

7. The catheter according to claim 1, wherein the catheter comprises two slidable members, whereby one slidable member consists of the second connector piece and a sheath having a longitudinal channel passing between a proximal end and a distal end, the sheath being joined to the second connector piece at the proximal end, and one slidable member is a ring member, whereby each at least one proximal attachment part of each of the at least one elongate member is attached to the ring member, whereby the ring member is arranged to be positioned between the second connector piece and the first connector piece and whereby the second connector piece is arranged to be positioned distal to the first connector piece.

8. The catheter according to claim 1, wherein each slidable member is arranged to be positioned distal to the first connector piece and the catheter comprises one elongate member having two proximal attachment parts, wherein the tubular body includes an inner lumen, an outer surface and two first openings passing between the outer surface and the inner lumen in the distal region, and wherein the distal coupling part is coupled to the distal region of the tubular body by passing through the two first openings.

9. The catheter according to claim 8, wherein the elongate member passes between the proximal attachment points and the two first openings outside the tubular body.

10. The catheter according to claim 8, wherein the tubular body further includes at least one second opening passing between the outer surface and the inner lumen, the at least one second opening being positioned proximal to the first openings, and at least one third opening passing between the outer surface and the inner lumen, the at least one third opening being positioned proximal to the at least one second opening, and wherein the elongate member passes between the first openings and the at least one second opening along the outer surface of the tubular body, between the at least one second opening and the at least one third opening along the inner lumen of the tubular body and between the at least one third opening and the proximal attachment points outside the tubular body.

11. The catheter according to claim 1, wherein the catheter comprises one slidable member, which slidable member is arranged to be positioned proximal to the first connector piece, and one elongate member having two proximal attachment parts, wherein the tubular body includes an inner lumen, an outer surface, two first openings passing between the outer surface and the inner lumen in the distal region and at least one second opening passing between the outer surface and the inner lumen, the at least one second opening being positioned proximal to the first openings, wherein the distal coupling part is coupled to the distal region of the tubular body by passing through the two first openings and wherein the elongate member passes between the first openings and the at least one second opening along the outer surface of the tubular body, between the at least one second opening and a proximal end of the tubular body along the inner lumen of the tubular body and between the proximal end of the tubular body and the proximal attachment points outside the tubular body.

12. The catheter according to claim 1, wherein the catheter comprises one slidable member, which slidable member is arranged to be positioned proximal to the first connector piece, and one elongate member having two proximal attachment parts, wherein the tubular body includes an inner lumen, an outer surface, two first openings passing between the outer surface and the inner lumen in the distal region, at least one second opening passing between the outer surface and the inner lumen, the at least one second opening being positioned proximal to the first openings, and at least one third opening passing between the outer surface and the inner lumen, the at least one third opening being positioned proximal to the at least one second opening, wherein the distal coupling part is coupled to the distal region of the tubular body by passing through the two first openings and wherein the elongate member passes between the first openings and the at least one second opening along the outer surface of the tubular body, between the at least one second opening and the at least one third opening along the inner lumen and between the at least one third opening and the proximal attachment points outside the tubular body.

13. The catheter according to claim 1, wherein the tubular body includes a loop positioned distal to each distal coupling part of each of the at least one elongate member.

14. The catheter according to claim 1, wherein the catheter comprises one elongate member and wherein the elongate member comprises one proximal attachment part.

15. The catheter according to claim 14, wherein the distal coupling part of the elongate member is attached to the outer surface of the tubular body in the distal region of the tubular body.

16. The catheter according to claim 1, wherein the catheter comprises two elongate members, whereby each elongate member comprises one proximal attachment part and one distal coupling part.

17. The catheter according to claim 16, wherein the distal coupling part of each elongate member is attached to the outer surface of the tubular body in the distal region of the tubular body.

18. A catheterization kit comprising a catheter according to claim 1, in which catheter each slidable member is arranged to be positioned distal to the first connector piece, and a split-away sheath being arranged to encircle at least a portion of the circumference of the tubular body and having a longitudinal channel passing between a proximal end and a distal end, the proximal end being arranged to be positioned outside the body and the distal end being arranged to be positioned inside the body, wherein the catheter is insertable into the split-away sheath.

19. A catheterization kit comprising a catheter according to claim 1 and a needle having an inner lumen.

20. A catheterization kit comprising a catheter according to claim 1 and a guide wire.

21. A catheterization kit comprising a catheter according to claim 1 and a dilator.

22. The catheterization kit according to claim 21, wherein the dilator comprises a radiopaque section.

23. A catheterization kit comprising a catheter according to claim 1 and a guiding pin to fit within the tubular body.

24. A catheterization kit comprising a catheter according to claim 1 and locking means for coupling the first connector piece and the second connector piece together.

25. A method of securing a catheter in a body cavity, the method comprising:
inserting a catheter into a body cavity, the catheter comprising a tubular body having a distal region and a proximal region; a first connector piece being permanently attached to the tubular body and being arranged to be positioned outside the body; at least one member being arranged to encircle at least a portion of the circumference of the tubular body and to be slidable along the tubular body, whereby each of the at least one slidable member is arranged to be positioned outside the body and one of the at least one slidable member comprises a second connector piece connectable to the first connector piece, and at least one elongate member, each of which having at least one proximal attachment part and a distal coupling part, the distal coupling part of each elongate member being coupled to the distal region of the tubular body and each of the at least one proximal attachment part being attached to one of the at least one slidable member at a proximal attachment point, whereby each elongate member extends from each respective proximal attachment point to the distal coupling part, whereby the slidable member to which each of the at least one proximal attachment part is attached is slidable to a position adjacent the first connector piece;
advancing the catheter into the body cavity until a distal end of the tubular body is positioned at a correct position within the body cavity;
stretching the at least one elongate member by adjusting the position of the second connector piece on the tubular body, and
bringing the first connector piece and the second connector piece together, whereby a loop is formed in the distal region of the tubular body.

26. The method according to claim 25 further comprising the step of:
locking the first connector piece to the second connector piece after a loop has been formed in the distal region of the tubular body.

27. The method according to claim 25, wherein the step of bringing the first connector piece and the second connector piece together comprises moving the second connector piece in the direction of the first connector piece.

28. The method according to claim 25, wherein each slidable member is arranged to be positioned distal to the first connector piece and the step of bringing the first connector piece and the second connector piece together comprises holding the second connector piece steady at a position along the tubular body where the elongate member is stretched and advancing the tubular body into the body cavity until the first connector piece is near the second connector piece.
